(19) United States
(12) Patent Application Publication
(10) Pub. No.: US 2023/0248990 A1
(43) Pub. Date: Aug. 10, 2023

(54) WIRE, TRANSCRANIAL MAGNETIC STIMULATION COIL AND TRANSCRANIAL MAGNETIC STIMULATOR FOR TRANSCRANIAL MAGNETIC STIMULATION

(71) Applicant: Shenzhen Yingchi Technology Co., Ltd., Xili Shenzhen, Guangdong (CN)

(72) Inventors: Feng XU, Xili Shenzhen, Guangdong (CN); Jingyan LONG, Xili Shenzhen, Guangdong (CN); Xinjie LIU, Xili Shenzhen, Guangdong (CN)

(21) Appl. No.: 18/015,779

(22) PCT Filed: Feb. 2, 2021
(86) PCT No.: PCT/CN2021/074748

§ 371 Date: Jan. 12, 2023

(30) Foreign Application Priority Data
Aug. 29, 2020 (CN) 202010890713.5

(51) Int. Cl. A61N 2/02; A61N 2/00; H01F 7/20; H01F 27/28

(52) U.S. Cl. CPC A61N 2/02; A61N 2/006; H01F 7/20; H01F 27/2823

(57) ABSTRACT

A wire for transcranial magnetic stimulation, which is used to fabricate a transcranial magnetic stimulation coil (100), comprising: an insulating tube and metal wires (20) sleeved on the exterior of the insulating tube (10); the insulating tube (10) is a hollow structure, and the interior of the insulating tube (10) is used for liquid or gas circulation, and the metal wires (20) or multi-stranded wire are used for energizing to thereby generate a magnetic field. The present invention may not only achieve the effect of cooling the stimulation coil (100), but also avoids the danger of the wire coil and the a circulating coolant making contact and conducting electricity.

### TECHNICAL FIELD

The present invention relates to the technical field of medical apparatus, especially a wire for transcranial magnetic stimulation, coil for transcranial magnetic stimulation and transcranial magnetic stimulation apparatus.

### BACKGROUND TECHNOLOGY

Transcranial magnetic stimulation is to generate pulse current by discharging a storage capacitor instantaneously into the stimulation coil, stimulate the stimulation coil to generate pulsed magnetic field, time-varying magnetic field induces electric field in the cortex, when the induction field exceeds the threshold of excitation of the brain nervous system, which resembles to excite the organism like electric stimulation, cortical stimulation effects can be reached.

Transcranial magnetic stimulation is primarily used in medical and scientific research areas, during treatment or researches, connect the stimulation coil with the transcranial magnetic stimulator, place the stimulation coil at the head of a patient with a central portion of the stimulation coil nestling on the part to be treated. During stimulation, the transcranial magnetic stimulation coil generates heat rapidly, a high quantity of heat is generated and conventional methods to cool down the transcranial magnetic stimulation coil comprise natural cooling, air cooling and liquid cooling.

Further, the liquid-cooling transcranial magnetic stimulation coil shall be connected with a liquid-cooling chassis for supplying circulating cooling liquid. Liquid-cooling transcanial magnetic cooling coils use primarily two cooling methods: the first one is to immerse the copper wire coil into circulating water and another is to reel the wire coil with a hollow copper duct with cooling liquid circulating in the copper duct. For the first method, a high requirement is given to waterproof performance of the casing, there is a high risk of liquid leakage, the wire coil is of a large volume and bulky and there is an electric conduction risk when the circulating liquid contacts directly the wire coil, and for the second method, there is a liquid leakage risk at where the wire coils of copper ducts are connected with the water ducts, it is difficult to reel the wire coil as the copper ducts are quite inflexible, and there is an electric conduction risk when the circulating liquid contacts directly the wire coil.

Therefore, it remains a problem in the field as to how to address the foregoing issues.

### SUMMARY OF THE INVENTION

The present invention provides a transcranial magnetic stimulation coil, aiming to address the foregoing issues.

According to a first aspect of the present invention, an embodiment of the present invention provides a wire for transcranial magnetic stimulation, wherein the wire for transcranial magnetic stimulation is configured to fabricate a transcranial magnetic stimulation coil, comprising: an insulating tube, and a metal conduit or a multi-stranded wire sleeved over the insulating tube; the insulating tube is configured to be hollow, a hollow portion of the insulating tube is configured for allowing liquid or air to circulate and flow therein, and the metal conduit or the multi-stranded wire is configured to be energized and thus generate a magnetic field. In the wire for transcranial magnetic stimulation, a cross section of the insulating tube is any of square, circular or oval.

In the wire for transcranial magnetic stimulation, the insulating tube is made from flexible materials.

According to a second aspect of the present invention, an embodiment of the present invention provides a transcranial magnetic stimulation coil, comprising a wire coil reeled from the wire for transcranial magnetic stimulation as stipulated above and a casing; wherein a shape of the casing is adaptive to a shape of the wire coil, and is configured for installing the wire coil.

In the transcranial magnetic stimulation coil according to the present invention, a shape of the wire coil is any of circular, splayed, conical, dual-conical and splayed with an included angle that is not flat.

In the transcranial magnetic stimulation coil according to the present invention, the casing is made from insulating materials.

According to a third aspect of the present invention, an embodiment of the present invention provides a transcranial magnetic stimulator, comprising the transcranial magnetic coil.

The technical solutions provided in the embodiments of the present invention can have the following beneficial effects: in the present invention, a wire for transcranial magnetic stimulation, a transcranial magnetic stimulation coil and a transcranial magnetic stimulator are designed, by which cooling of stimulation coils can be done in the meanwhile the risk of electric conduction due to contact between the coil and the circulating cooling liquid is avoided.

### BRIEF DESCRIPTION OF DRAWINGS

To better illustrate the technical solutions in the embodiments of the present invention more clearly, drawings to be used in the description of the embodiments are briefly introduced herein, apparently, the drawings in the following description are only some embodiments of the present invention, for those of ordinary skill in the art, without paying creative effort, it is possible to obtain other drawings based on these drawings.

FIG. 1 is a sectional diagram showing the wire for transcranial magnetic stimulation in an embodiment of the present invention; and

FIG. 2 is a structural diagram showing the transcranial stimulation coil according to an embodiment of the present invention.

Markups in the drawings are:

100, transcranial magnetic stimulation coil; 10, insulation tube; and 20 metal conduit.

### EMBODIMENTS

Hereinafter a clear and complete description will be given to the technical solutions of the present invention based on the drawings of the embodiments of the present invention, apparently the embodiments described here are only some of the embodiments of the present invention rather than all. Based on the embodiments of the present invention, all other embodiments obtained by those of ordinary skill in the art without paying creative effort fall into the protection scope of the present invention.

It shall be comprehensible that, terms used in the description of the present invention are only intended to describe the embodiment rather than limit the present invention. For example, unless clearly indicated otherwise, “a”, “an” and “the” in singular forms used in the specification and the appended claims intend to cover plural forms.

It shall be further understood that, the term “and/or” used in the specification and the appended claims refers to any combination and all possible combination of one or more of the listed items and include the combinations.

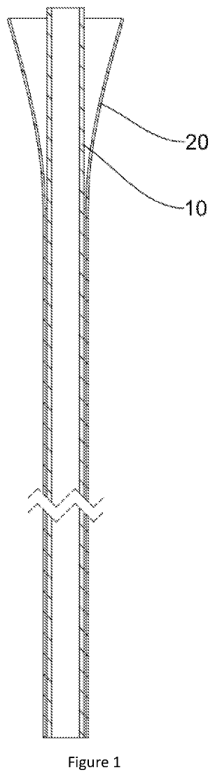

Please refer to FIG. 1, the present invention discloses a wire for transcranial magnetic stimulation, comprises an insulation tube 10 and a metal conduit 20 sleeved over the insulation tube. Wherein the insulation tube 10 is a hollow structure, and the hollow portion in the insulation tube 10 is configured for flowing of liquids or gases. The wire disclosed in the present invention is configured to make a transcranial magnetic stimulation coil 100. The metal conduit 20 sleeved over the insulation tube 10 is configured to be energized and generate magnetic field for conducting transcranial magnetic stimulation. When being energized, the metal conduit 20 will generate a lot of heat, by circulation and flowing of liquids or gases in the insulation tube 10 heat in the metal conduit 20 or multi-stranded wire can be disseminated. In the present invention, continuous cooling of the stimulation coil is realized while contact of the metal conduit 20 or the multi-stranded wire with the cooling liquid is avoided, thus safety performance of the transcranial magnetic stimulation coil is improved. Furthermore, as there is no coupling piece in the insulation tube, the risk of liquid leakage can be reduced, and it is no longer necessary to immerse the transcranial magnetic coil in circulating cooling liquids.

In the present embodiment, the metal conduit 20 is a single-stranded wire or a multi-stranded wire. For example, the metal conduit 20 can be an insulated metal braided conduit, bare metal braided conduit, multi-stranded insulated metal wire and multi-stranded bare metal wires. In some embodiments, when the metal conduit is a bare metal wire, an insulating layer can be encapsulated thereon.

In an optional embodiment, a cross section of the insulating tube 10 is any of rectangular, circular and oval. In the present embodiment, there is no limitation on a cross section shape of the insulation tube 10 for the purpose of entering circulating cooling liquid or circulating cooling air, the insulation tube 10 can also be configured to be other irregular shapes, as long as magnetic field can be generated when the wire is reeled to be the coil.

In an optional embodiment, the insulation tube 10 is made from flexible materials, and by making the wire with flexible materials, reeling difficulty of the coil is reduced and weight of the coil can be reduced.

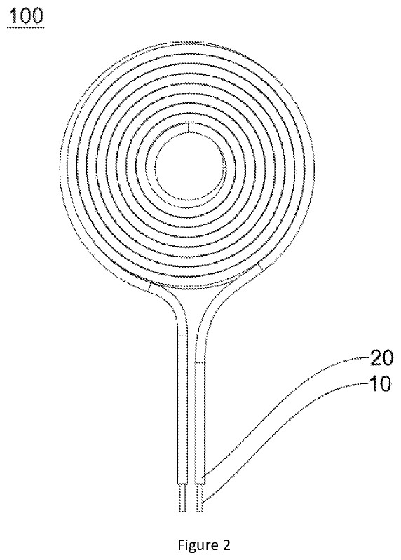

Please refer to FIG. 2, the present invention further protects a transcranial magnetic stimulation coil 100, comprising a wire coil reeled with the wire for transcranial magnetic stimulation and a casing (not shown in the drawings), a shape of the casing is adaptive to a shape of the wire coil, and configured for housing the wire coil.

In an optional embodiment, a shape of the wire coil is any of circular, splayed, conical, dual-conical and splayed shape with an included angle. To reel the wire coil into these shapes is to generate a magnetic field, apparently it is possible to configure the wire coil to be other shapes that can generate magnetic field. In the present embodiment, the casing is made from insulating materials, and by using the insulating materials the safety performance of the transcranial magnetic stimulation coil according to the present invention is higher.

The present invention further provides a transcranial magnetic stimulator, comprising the transcranial magnetic stimulation coil. The transcranial magnetic stimulator can realize stimulation coil cooling effects while avoid the risk of electric conduction when the wire coil contact the circulating cooling liquid (as the wire coil is reeled from a single wire, no coupling component is present, therefore, there is no risk of liquid leakage). A volume of the casing can be reduced significantly, and the insulating tube is flexible and light, in this way the reeling difficulty and the weight of the wire coil is greatly reduced.

The foregoing are only some specific embodiments of the present invention, however, the protection scope of the present invention is not restricted to these embodiments disclosed herein, a variety of equivalent replacement and modifications can be done by those skilled in the art within the scope disclosed in the present invention, and the equivalent replacement and modifications shall be covered in the protection scope of the present invention. Therefore, the protection scope of the present invention shall be defined only by the protection scope of the claims.

1. A wire for transcranial magnetic stimulation, configured for making a transcranial magnetic stimulation coil, comprising: an insulating tube and a metal conduit sleeved over the insulating tube; the insulating tube is a hollow structure, an inner portion of the insulation tube is configured for circulation and flowing of liquids or gases, and the metal conduit or a multi-stranded wire is configured to be energized and generate a magnetic field.

2. The wire for transcranial magnetic stimulation, wherein a cross section of the insulating tube is any of rectangular, circular and oval.

3. The wire for transcranial magnetic stimulation, wherein the insulating tube is made from flexible materials.

4. A transcranial magnetic stimulation coil, comprises a casing and a wire coil reeled with the wire for transcranial magnetic stimulation as defined in claim 1; a shape of the casing is adaptive to a shape of the wire coil and the casing is configured for housing the wire coil.

5. The transcranial magnetic stimulation coil according to claim 4, a shape of the wire coil is any of circular, splayed, conical, dual-conical and splayed shape with an included angle.

6. The transcranial magnetic stimulation coil according to claim 4, wherein the casing is made from insulating materials.

7. A transcranial magnetic stimulator, comprises the transcranial stimulation coil as defined in claim 4.
